(19) United States
(12) Patent Application Publication
(10) Pub. No.: US 2026/0224102 A1
(43) Pub. Date: Aug. 6, 2026

(54) MULTIPLE-CHANNEL IMAGING LAPAROSCOPY AND USES THEREOF

(71) Applicant: CHILDREN'S NATIONAL MEDICAL CENTER, Washington, DC (US)

(72) Inventors: Richard Jaepyeong CHA, Washington, DC (US); Bo NING, Washington, DC (US)

(73) Assignee: CHILDREN'S NATIONAL MEDICAL CENTER, Washington, DC (US)

(21) Appl. No.: 19/159,803

(22) PCT Filed: Mar. 11, 2024
(86) PCT No.: PCT/US2024/019361

§ 371 Date: Aug. 26, 2025

Related U.S. Application Data
(60) Provisional application No. 63/489,605, filed on Mar. 10, 2023.

(51) Int. Cl. A61B 1/313; A61B 1/04; A61B 1/05; A61B 1/06

(52) U.S. Cl. CPC A61B 1/3132; A61B 1/042; A61B 1/05; A61B 1/0669

(57) ABSTRACT

An endoscopic device, including an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a laser pathway shifter between the laser diode and the proximal end of the illumination channel and configured to change a propagation direction of the laser; and an imaging pathway shifter between the proximal end of the imaging channel and the at least one imaging sensor and configured to change a propagation direction of light from the imaging channel.

### CROSS-REFERENCE TO RELATED APPLICATIONS

The present application claims priority to U.S. Provisional Application No. 63/489,605, filed Mar. 10, 2023, which is incorporated herein by reference in its entirety for all purposes.

### FEDERAL FUNDING DISCLOSURE

This disclosure was made with government support under Grant Number DK131650 awarded by the National Institutes of Health. The government has certain rights to the disclosure.

### BACKGROUND

### Field of the Disclosure

The present disclosure relates to endoscopic devices for imaging the body.

### Description of the Related Art

Laser speckle contrast imaging (LSCI) can be used in endoscopy to assess blood flow. Laparoscopy is a type of endoscopy wherein a scope (laparoscope) is inserted into the body through a small incision, typically at the abdomen or pelvis. Laparoscopy can enable diagnosis and therapeutic intervention (e.g., surgery) via minimally invasive access to the internal organs.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to an endoscopic device, comprising an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a laser pathway shifter between the laser diode and the proximal end of the illumination channel and configured to change a propagation direction of the laser; and an imaging pathway shifter between the proximal end of the imaging channel and the at least one imaging sensor and configured to change a propagation direction of light from the imaging channel, wherein the illumination channel is configured to transmit the laser from the laser diode to a distal end of the illumination channel, and wherein the imaging channel is configured to transmit light from a distal end of the imaging channel to the at least one imaging sensor.

In one embodiment, the present disclosure is related to an endoscopic device, comprising: an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a first pair of prisms between the laser diode and the proximal end of the illumination channel and configured to change a propagation direction of the laser; and a second pair of prisms between the proximal end of the imaging channel and the at least one imaging sensor and configured to change a propagation direction of light from the imaging channel, wherein the illumination channel is configured to transmit the laser from the laser diode to a distal end of the illumination channel, and wherein the imaging channel is configured to transmit light from a distal end of the imaging channel to the at least one imaging sensor.

In one embodiment, the present disclosure is related to an endoscopic device, comprising: an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a first pair of prisms between the laser diode and the proximal end of the illumination channel and configured to change a propagation direction of the laser; and a second pair of prisms between the proximal end of the imaging channel and the at least one imaging sensor and configured to change a propagation direction of light from the imaging channel, wherein the illumination channel is configured to transmit the laser from the laser diode to a distal end of the illumination channel, wherein the imaging channel is configured to transmit light from a distal end of the imaging channel to the at least one imaging sensor, and wherein the at least one imaging sensor includes a near infrared (NIR) sensor.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an illustration of an endoscopic device, according to one embodiment of the present disclosure;

FIG. 2 is an illustration of light propagation through an endoscopic device, according to one embodiment of the present disclosure;

FIG. 3 is an illustration of an endoscopic device, according to one embodiment of the present disclosure;

FIG. 4 is an illustration of an endoscopic device, according to one embodiment of the present disclosure;

FIG. 5 is an illustration of an endoscopic image, according to one embodiment of the present disclosure; and

FIG. 6 is an illustration of an endoscopic image, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed to an endoscopic device configured for laser speckle contrast imaging (LSCI). The endoscopic device can be inserted into a body via an opening, the opening including an orifice or an incision. LSCI can be used to determine and analyze blood flow (perfusion) in the body. In one embodiment, the endoscopic device can be used for various surgical procedures to provide real-time perfusion data. Surgical procedures can include, but are not limited to, minimally invasive procedures, tumor resection, gastrointestinal surgery, urological procedures, neurosurgery and neuroendoscopy, vascular surgery, reconstructive surgery (e.g., tissue transfer), orthopedic surgery (e.g., joint reconstruction, bone grafting), and gynecological surgery (e.g., ovarian cyst removal, endometriosis treatment). In general, real-time blood flow data and imaging of the body can be used to minimize tissue damage, identify and preserve critical blood vessels, differentiate veins from arteries (e.g., with their different blood flow rates) and reduce the risk of ischemic events during surgery.

LSCI is an imaging modality that uses the speckle pattern of light to determine blood flow in body tissue. LSCI is light-based and does not require the use of contrast agents or dyes, such as indocyanine green. A laser can illuminate a surface (tissue). The coherence of laser light can result in interference between wavefronts traveling in the same direction. The interference results in fluctuations in the scattered reflection of the light off of the surface, resulting in speckle (“speckle pattern,” “speckle noise”).

In medical imaging, the light from the laser can reflect off of the surfaces of blood cells to create a speckle pattern of light. The speckle pattern can be received by one or more imaging sensors. Speckle contrast can be defined as the ratio of the standard deviation of speckle intensity to the mean speckle intensity in a spatial or temporal (or spatiotemporal) window. The movement of the blood cells as blood flows through the body causes the speckle pattern to fluctuate and blur, thereby decreasing the speckle contrast. Change in speckle contrast over time can be used to determine the velocity of blood flow in the surface illuminated by the laser.

In one embodiment, the endoscopic device of the present disclosure includes a first channel and a second channel configured for LSCI. The first channel can include the laser light source. The second channel can include one or more imaging sensors configured to acquire speckle patterns resulting from illumination by the laser of the first channel. Light from the laser light source and light reaching the imaging sensors can be separated into the channels, resulting in increased robustness, consistency, and speckle pattern quality. The use of two separate channels can also improve light coupling efficiency of the laser and can reduce interference in both the illumination and the imaging of the surface.

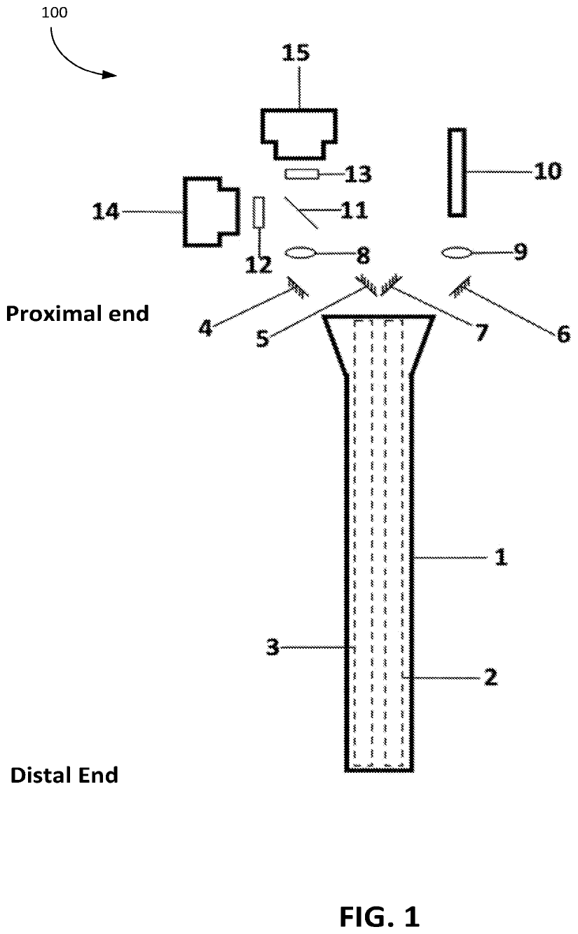

FIG. 1 is an illustration of an endoscopic device 100 according to one embodiment. In one embodiment, the endoscopic device 100 can be a laparoscope. The distal end of the endoscopic device 100 can be configured to be inserted into the body via an incision. The endoscopic device 100 can include or be coupled with a proximal handle or navigational controller. The endoscopic device can include an outer body 1, the outer body 1 surrounding at least a first channel 2 and a second channel 3. The outer body can also be referred to as the scope. The scope can form a scope channel containing the first channel and the second channel. The scope can be rigid and can include lens relays to prevent interference (e.g., despeckling) that can be caused by flexing of fibers (e.g., fiber-bundle light guides) of the channels. The distal end of each of the first channel 2 and the second channel 3 can terminate in an opening or a transparent face. In one embodiment, the first channel 2 can be an illumination channel and can deliver light from at least one laser source 10 to the distal end of the channel 2. The second channel 3 can be an imaging channel and can direct light from the distal end of the channel 3 to one or more imaging sensors 14, 15. In one embodiment, the illumination channel 2 and the imaging channel 3 can be approximately cylindrical, with a diameter of approximately 0.1 millimeters (mm) to 100 mm. In one embodiment, the diameter of the channels can be less than 0.1 mm or greater than 100 mm. The illumination channel 2 and the imaging channel 3 can be parallel and can have separate longitudinal axes. In one embodiment, as illustrated in FIG. 1, the illumination channel 2 and the imaging channel 3 are not coaxial, and the channels are not contained within each other.

In one embodiment, the endoscopic device 100 can include more than two channels. For example, a second illumination channel (not pictured) can deliver light from a second laser. Additionally or alternatively, a second imaging channel (not pictured) can direct light to a second set of imaging sensors. In one embodiment, the endoscopic device 100 can include one or more working channels in addition to the illumination channel 2 and the imaging channel 3.

In one embodiment, the laser source 10 can be a single-mode fiber coupled laser diode. In one embodiment, the laser can be a low-power laser. In one embodiment, the power to the laser diode can be approximately 60 milliwatts (mW). In one embodiment, the power to the laser diode can be approximately 250 mW. In one embodiment, the power to the laser diode can be within the range of approximately 1 mW to 100 watts (W). In one embodiment, the laser emitted by the laser source 10 can have a wavelength in the near-red to infrared range, e.g., 658 nanometers (nm), 660 nm. In one embodiment, the wavelength of the laser can be between 200 nm to 2000 nm. In one embodiment, the endoscopic device 100 can include more than one light source. For example, the endoscopic device 100 can include a first laser diode of a first wavelength and a second laser or light-emitting diode of a second wavelength.

In one embodiment, the endoscope 100 can include at least one lens 9 between the laser source 10 and the distal end of the illumination channel 2. The at least one lens 9 can diverge the laser from the laser source 10 to control the diameter of the laser as emitted at the distal end of the illumination channel 2. In one embodiment, the at least one lens 9 can include a plano-convex lens. In one embodiment, the at least one lens 9 can be a combination of lenses. In one embodiment, the laser can be emitted at the distal end of the illumination channel without the at least one lens when the diameter of the laser as emitted from the laser source 10 is within a certain range. The range of diameters of the laser can depend on the diameter of the outer body 1 and the illumination channel 2.

In one embodiment, the endoscope 100 can include at least one prism (e.g., a triangular prism) between the laser source 10 and the illumination channel 2. In one embodiment, the at least one prism can include a first prism pair 6, 7. The first prism pair can direct light from the laser source 10 into the illumination channel 2. For example, the laser source 10 can emit a vertical laser beam. The first prism 6, located below the laser source 10, can redirect the propagation direction of the vertical laser beam to a horizontal direction. The second prism 7 can be horizontally aligned with the first prism 6 and can redirect the horizontal laser beam to a vertical direction and into the illumination channel 2. In this manner, the laser source 10 can be vertically displaced from the illumination channel 2 rather than being in line with the illumination channel 2. In one embodiment, the at least one prism can be a single mirror or a mirror pair.

In one embodiment, the endoscope 100 can include at least one imaging sensor. In one embodiment, the at least one imaging sensor can include a first imaging sensor 14 and a second imaging sensor 15. The at least one imaging sensor can receive light (optical signals) from the distal end of the imaging channel 3. The endoscopic device can include or be coupled to processing circuitry configured to process the received light. In one embodiment, the processing circuitry can be configured to process speckle patterns in the received light for LSCI. In one embodiment, the at least one imaging sensor can be a complementary metal-oxide-semiconductor (CMOS) sensor or a charge-coupled device (CCD). In one embodiment, the at least one imaging sensor can be used to detect light of different wavelengths, including visible and non-visible light. For example, the first imaging sensor 14 can be a red-green-blue (RGB) sensor (e.g., CMV4000 RGB) and the second imaging sensor 15 can be a near-infrared (NIR) sensor (e.g., CMV4000 NIR sensor, Sony IMX178 NIR sensor). Additional or alternative imaging sensors can be included for additional or alternative imaging modalities.

Light can be directed through the imaging channel 3 to the at least one imaging sensor 14, 15. In one embodiment, the endoscope 100 can include at least one prism (e.g., a triangular prism) between the imaging channel 3 and the at least one imaging sensor. In one embodiment, the at least one prism can include a second prism pair 4, 5. The second prism pair can direct light from the proximal end of the imaging channel 3 to the at least one imaging sensor. For example, light emerging from the proximal end of the imaging channel 3 can be traveling in a vertical direction. The first prism 5, located above the proximal end of the imaging channel 3, can redirect the propagation direction of the light to a horizontal direction. The second prism 4 can be horizontally aligned with the first prism 5 and can redirect the horizontally propagating light to a vertical direction and into the at least one imaging sensor. In this manner, the at least one imaging sensor and associated elements can be vertically displaced from the imaging channel 3 rather than being in line with the imaging channel 3. In one embodiment, the at least one prism can be a single mirror a mirror pair.

In one embodiment, the endoscope 100 can include at least one imaging lens 8 between the proximal end of the imaging channel 3 and the at least one imaging sensor. The at least one imaging lens 8 can be configured to focus light from the imaging channel 3. In one embodiment, the at least one imaging lens 8 can be a single lens configured to focus light that is transmitted to a plurality of imaging sensors, as illustrated in FIG. 1. In one embodiment, the endoscope can include an imaging lens for each imaging sensor. Any combination and number of imaging lenses and imaging sensors is compatible with the present disclosure.

In one embodiment, the endoscope can include at least one beam splitter 11 (e.g., a prism) located between the imaging channel 3 and the at least one imaging sensor. The prism can be a plate or a cube. The at least one beam splitter 11 can split and direct the light from the imaging channel 3 to each imaging sensor. In one embodiment, the at least one beam splitter 11 can be a non-polarizing beam splitter. For example, a non-polarizing beam splitter can be configured for 70% transmission and 30% reflection. In one embodiment, the at least one beam splitter 11 can include a filter, such as a long-pass filter with a cutoff frequency of approximately 650 nm. In one embodiment, the at least one beam splitter 11 can be a dichroic material. In one embodiment, the at least one beam splitter 11 can be polarizing. In one embodiment, the at least one beam splitter 11 can include a combination or series of beam splitters.

In one embodiment, the endoscopic device 100 can include at least one filter located between the imaging channel 3 and the at least one imaging sensor. The at least one filter can include a combination or series of filters. In one example, the endoscopic device 100 can include a first filter 12 between the imaging channel 3 and the first imaging sensor 14 and a second filter 13 between the imaging channel 3 and the second imaging sensor 15, as illustrated in FIG. 1. In one embodiment, the at least one filter can include a polarizer. The at least one filter can include a bandpass filter, notch filter, high-pass filter, low-pass filter, etc. In one example, the first filter 12 can be a notch filter, and the second filter 13 can be a combination of a bandpass filter and a polarizing filter. The composition of the filter can be based on the at least one imaging sensor, the laser, and/or the surface being imaged.

In one embodiment, the laser source 10 can be located at or near the distal end of the illumination channel 2 (e.g., in the illumination channel). In one embodiment, the lens 9 can be located between the laser source 10 and the distal opening of the illumination channel 2. In one embodiment, the at least one imaging sensor can be located at or near the distal end of the imaging channel 3 (e.g., in the imaging channel). In one embodiment, the imaging lens 8 and a filter and/or polarizer can be located between the distal opening of the imaging channel 3 and the at least one imaging sensor.

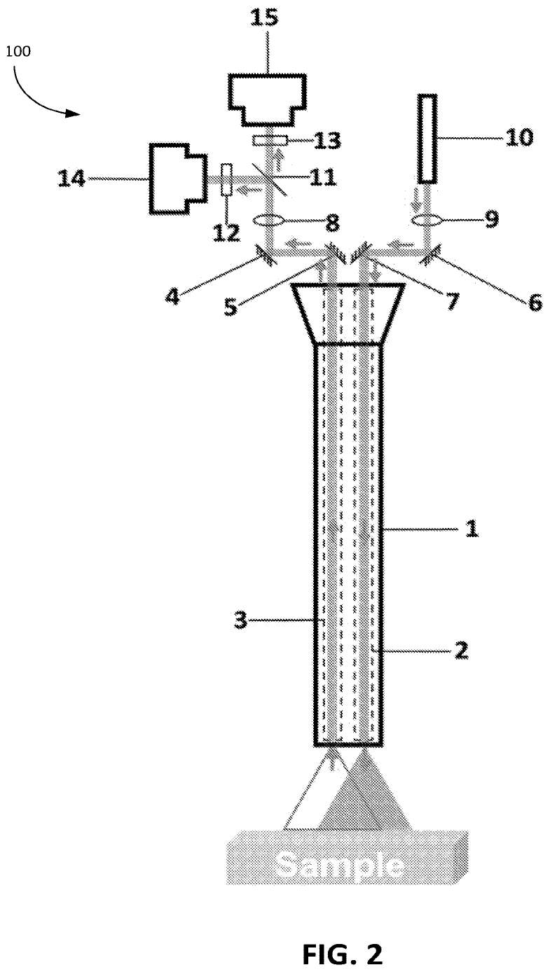

FIG. 2 is an illustration of the passage of light through the endoscopic device 100.

The distal end of the endoscopic device 100 can be directed at a sample, such as body tissue. The laser emitted by the laser source 10 can be diverged by the lens 9 and directed by the first prism pair 6, 7 into the illumination channel 2. The laser can then be output at the distal end of the illumination channel 2. The illumination channel 2 being independent from the imaging channel 3 can result in more efficient and undisturbed transmittal of the laser. The imaging components do not interfere with the transmittal of light through the illumination channel 2 and the distal opening. Similarly, the emitted laser does not interfere with the light that is transmitted through the imaging channel 3.

The laser emitted by the illumination channel 2 can create a speckle pattern on the sample. The light of the speckle pattern can travel through the imaging channel 3 to the second prism pair 5, 4. The light can be directed by the second prism pair 5, 4 through the imaging lens 8 and to each of the imaging sensors 14, 15. The light can be filtered, polarized, and/or split prior to reaching the imaging sensors 14, 15. The imaging sensors 14, 15 can then generate an image of the sample. The laser source 10 can continue illuminating the sample, and the imaging sensors 14, 15 can receive light in order to image the sample over time. Changes in the speckle pattern over time can be used to determine a velocity of blood flow in the sample.

In one embodiment, the illumination channel 2 and/or the imaging channel 3 can include one or more relay lenses (lens relays). The one or more relay lenses can extend or manipulate the optical path along the length of the channel. In one embodiment, the relay lenses can include rod lenses, e.g., Hopkins rod lenses. The relay lenses can include achromatic lenses, gradient-index of refraction lenses, etc. In one embodiment, the illumination channel 2 and/or the imaging channel 3 can be coated with an anti-reflective coating to prevent crosstalk between the channels. The anti-reflective coating can be a near-infrared anti-reflective coating. In one embodiment, the channels can include light shields surrounding an opening of each channel.

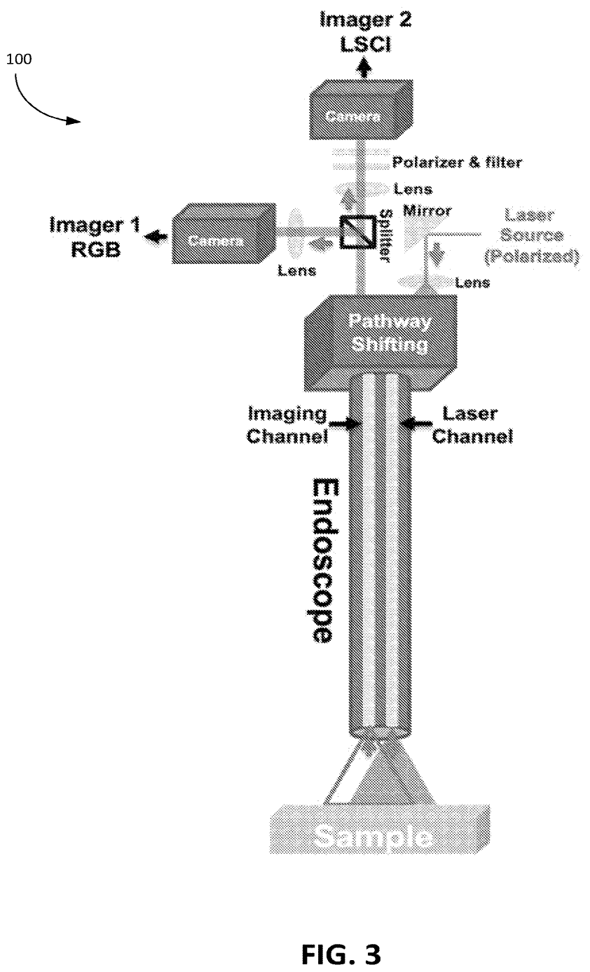

FIG. 3 is an illustration of an endoscopic device according to one embodiment. The endoscopic device can include an illumination channel (laser channel), laser source, and laser manipulation elements (e.g., prism, mirror, lens, polarizer, filter) as described with reference to FIG. 1. Light from the laser source can be directed into the laser channel via a laser pathway shifter. The laser pathway shifter can include, for example, the first prism pair 6, 7 of FIG. 1 or one or more mirrors. In one embodiment, the laser source can emit polarized light. The endoscopic device can include an RGB imager (Imager 1) and an LSCI imager (Imager 2). Light transmitted through the imaging channel can be directed to imaging elements via an imaging pathway shifter. The imaging pathway shifter can include, for example, the second prism pair 4, 5 of FIG. 1 or one or more mirrors. In one embodiment, the pathway shifters can include a light sensor and emitter pair. The light transmitted through the imaging channel can be split by a beam splitter and directed to the RGB imager (camera) and the LSCI imager (camera). The light transmitted through the imaging channel can be manipulated by one or more light manipulation elements (e.g., prism, mirror, lens, polarizer, filter) prior to being transmitted to the imagers. The combination of the RGB imager and the LSCI imager can be used to generate images of blood perfusion in the sample. In one embodiment, the endoscopic device can be used for real-time determination of blood flow in the sample while the sample is being imaged.

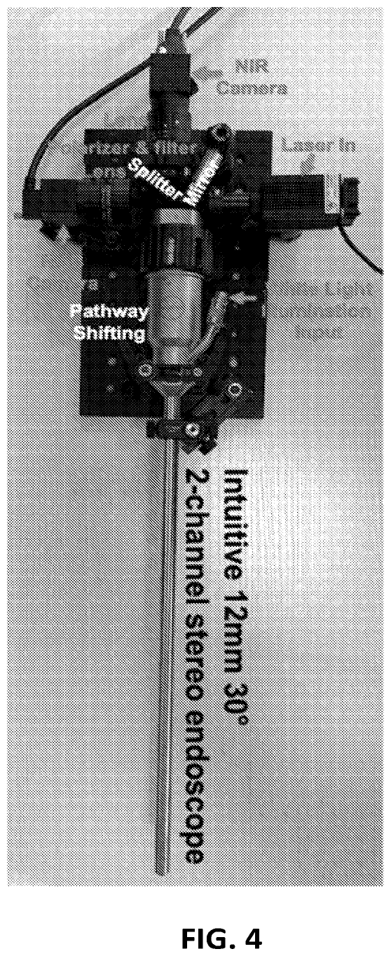

FIG. 4 is an illustration of an endoscopic device according to one embodiment. The scope can be a 2-channel stereo scope containing the illumination channel and the imaging channel. The scope can have a diameter of 12 mm. In one embodiment, the distal opening of the scope can be angled, e.g., have a 30° angle relative to the cross-section of the body. In one embodiment, the scope can be curved or bent. The separate illumination and imaging channels and the corresponding prism pairs can enable the laser source and the imaging sensors to be displaced from the central axis of the scope. The laser source and the imaging sensors can thereby occupy a larger footprint than the diameter of the scope. The arrangement of the laser source and the imaging sensors can enable the inclusion of robust light manipulation elements and imaging sensors, including imaging sensors of different imaging modalities, that are not limited by the size and position of the scope. The separation of the illumination channel and the imaging channel can also enable the inclusion of separate lenses and prism pairs for the emitted laser and the received light. Therefore, the light manipulation elements for each channel are not shared and do not interfere with light from the other channel.

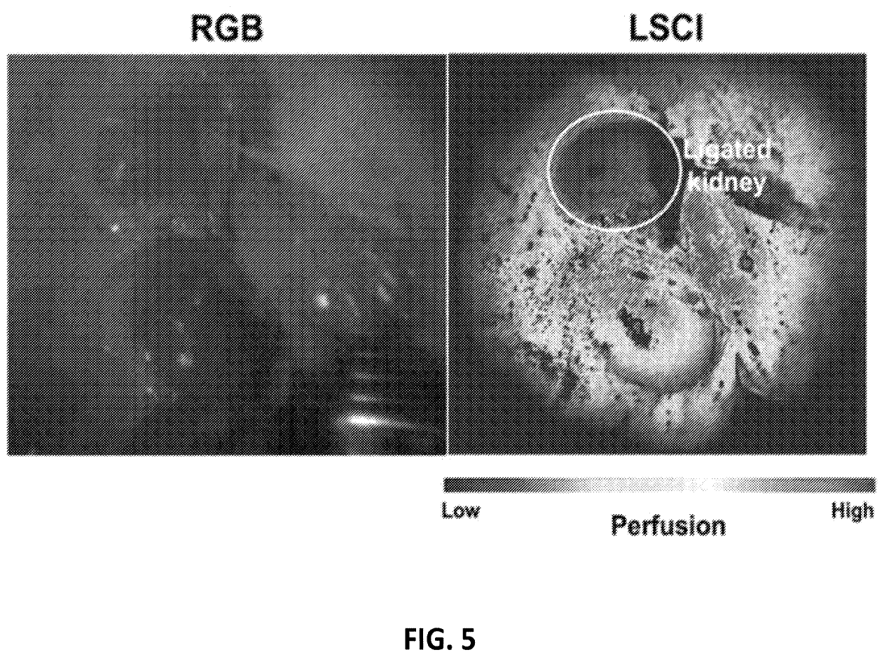

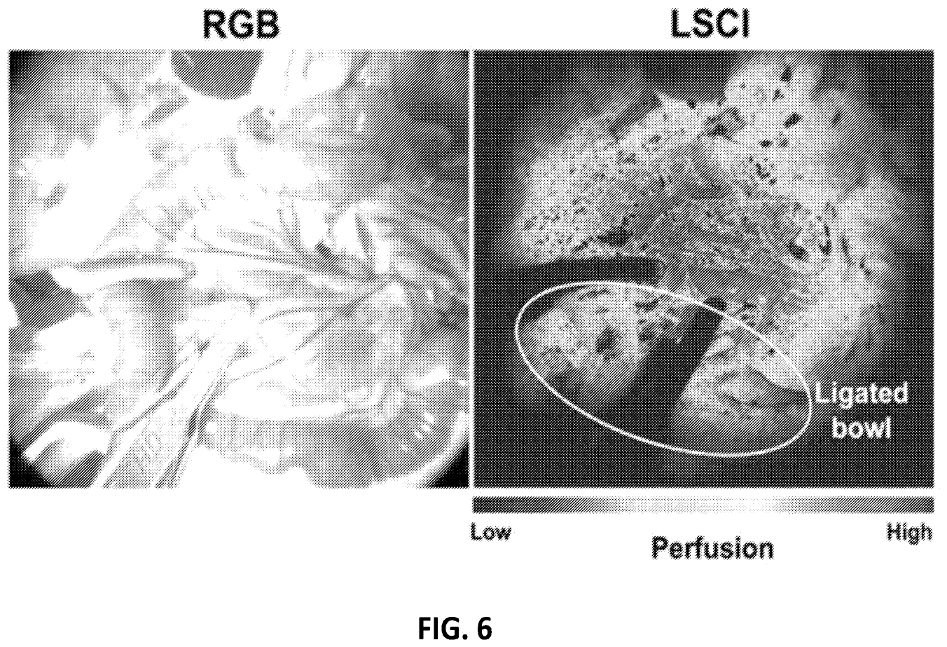

FIG. 5 illustrates an RGB image and an LSCI image of a sample that are generated based on imaging data generated by the endoscopic device according to one embodiment. The LSCI image can indicate perfusion in the sample based on the varying speckle pattern that is imaged by the endoscopic device. The LSCI image accurately indicates that there is low blood flow in a region of the sample. The region having low blood flow is a ligated region of the kidney where blood flow is cut off by a band, as can be seen in the RGB image. FIG. 6 illustrates an RGB image and an LSCI image of a sample that are generated based on imaging data generated by the endoscopic device according to one embodiment. The LSCI image can indicate perfusion in the sample based on the varying speckle pattern that is imaged by the endoscopic device. The LSCI image accurately indicates that there is low blood flow in a region of the sample. The region having low blood flow is a ligated region where blood flow is cut off by two clamps, as can be seen in the RGB image. The endoscopic device can include the RGB imager and the LSCI imager used to generate both images in real-time for more robust medical assessment.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:
(1) An endoscopic device, comprising an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a laser pathway shifter between the laser diode and the proximal end of the illumination channel and configured to change a propagation direction of the laser; and an imaging pathway shifter between the proximal end of the imaging channel and the at least one imaging sensor and configured to change a propagation direction of light from the imaging channel, wherein the illumination channel is configured to transmit the laser from the laser diode to a distal end of the illumination channel, and wherein the imaging channel is configured to transmit light from a distal end of the imaging channel to the at least one imaging sensor.
(2) The endoscopic device of (1), wherein the at least one imaging sensor includes a near-infrared (NIR) imaging sensor.
(3) The endoscopic device of (1) to (2), further comprising processing circuitry configured to identify variations in speckle patterns in the light received by the at least one imaging sensor, or transmission circuitry configured to transmit data corresponding to the light received by the at least one imaging sensor to external processing circuitry configured to identify the variations in the speckle patterns in the light received by the at least one imaging sensor.
(4) The endoscopic device of (1) to (3), wherein the at least one imaging sensor includes a red-green-blue (RGB) imaging sensor.
(5) The endoscopic device of (1) to (4), wherein the at least one imaging sensor includes a near-infrared (NIR) imaging sensor and a red-green-blue (RGB) imaging sensor.
(6) The endoscopic device of (1) to (5), wherein the imaging pathway shifter includes a beam splitter between the proximal end of the imaging channel and the at least one imaging sensor.
(7) The endoscopic device of (1) to (6), wherein the laser pathway shifter includes at least one prism.
(8) The endoscopic device of (1) to (7), wherein the laser pathway shifter includes at least one mirror.
(9) The endoscopic device of (1) to (8), wherein the imaging pathway shifter includes at least one prism.
(10) The endoscopic device of (1) to (9), wherein the imaging pathway shifter includes at least one mirror.
(11) The endoscopic device of (1) to (10), wherein the illumination channel and the imaging channel are contained in a rigid scope channel and the illumination channel and/or the imaging channel includes at least one relay lens.
(12) An endoscopic device, comprising: an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a first pair of prisms between the laser diode and the proximal end of the illumination channel and configured to change a propagation direction of the laser; and a second pair of prisms between the proximal end of the imaging channel and the at least one imaging sensor and configured to change a propagation direction of light from the imaging channel, wherein the illumination channel is configured to transmit the laser from the laser diode to a distal end of the illumination channel, and wherein the imaging channel is configured to transmit light from a distal end of the imaging channel to the at least one imaging sensor.
(13) The endoscopic device of (12), wherein the at least one imaging sensor includes a near-infrared (NIR) imaging sensor.
(14) The endoscopic device of (12) to (13), wherein the at least one imaging sensor includes a red-green-blue (RGB) imaging sensor.
(15) The endoscopic device of (12) to (14), wherein the at least one imaging sensor includes a near-infrared (NIR) imaging sensor and a red-green-blue (RGB) imaging sensor.
(16) The endoscopic device of (12) to (15), further comprising a beam splitter between the second pair of prisms and the at least one imaging sensor.
(17) An endoscopic device, comprising: an illumination channel; an imaging channel parallel to the illumination channel and having a different longitudinal axis from a longitudinal axis of the illumination channel; a laser diode at a proximal end of the illumination channel and offset from the longitudinal axis of the illumination channel and configured to emit a laser; at least one imaging sensor at a proximal end of the imaging channel and offset from the longitudinal axis of the imaging channel; a first pair of prisms between the laser diode and the proximal end of the illumination channel and configured to change a propagation direction of the laser; and a second pair of prisms between the proximal end of the imaging channel and the at least one imaging sensor and configured to change a propagation direction of light from the imaging channel, wherein the illumination channel is configured to transmit the laser from the laser diode to a distal end of the illumination channel, wherein the imaging channel is configured to transmit light from a distal end of the imaging channel to the at least one imaging sensor, and wherein the at least one imaging sensor includes a near infrared (NIR) sensor.
(18) The endoscopic device of (17), wherein the at least one imaging sensor further includes a red-green-blue (RGB) imaging sensor.
(19) The endoscopic device of (17) to (18), further comprising a beam splitter between the second pair of prisms and the at least one imaging sensor.
(20) The endoscopic device of (17) to (19), further comprising processing circuitry configured to identify variations in speckle patterns in the light received by the at least one imaging sensor, or transmission circuitry configured to transmit data corresponding to the light received by the at least one imaging sensor to external processing circuitry configured to identify the variations in the speckle patterns in the light received by the at least one imaging sensor.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. An endoscopic device, comprising:
an illumination channel;
an imaging channel parallel to the illumination channel and having a different longitudinal axis from a longitudinal axis of the illumination channel;
a laser diode at a proximal end of the illumination channel and offset from the longitudinal axis of the illumination channel and configured to emit a laser;
at least one imaging sensor at a proximal end of the imaging channel and offset from the longitudinal axis of the imaging channel;
a laser pathway shifter between the laser diode and the proximal end of the illumination channel and configured to change a propagation direction of the laser; and
an imaging pathway shifter between the proximal end of the imaging channel and the at least one imaging sensor and configured to change a propagation direction of light from the imaging channel,
wherein the illumination channel is configured to transmit the laser from the laser diode to a distal end of the illumination channel, and
wherein the imaging channel is configured to transmit light from a distal end of the imaging channel to the at least one imaging sensor.

2. The endoscopic device of claim 1, wherein the at least one imaging sensor includes a near-infrared (NIR) imaging sensor.

3. The endoscopic device of claim 2, further comprising
processing circuitry configured to identify variations in speckle patterns in the light received by the at least one imaging sensor, or
transmission circuitry configured to transmit data corresponding to the light received by the at least one imaging sensor to external processing circuitry configured to identify the variations in the speckle patterns in the light received by the at least one imaging sensor.

4. The endoscopic device of claim 1, wherein the at least one imaging sensor includes a red-green-blue (RGB) imaging sensor.

5. The endoscopic device of claim 1, wherein the at least one imaging sensor includes a near-infrared (NIR) imaging sensor and a red-green-blue (RGB) imaging sensor.

6. The endoscopic device of claim 5, wherein the imaging pathway shifter includes a beam splitter between the proximal end of the imaging channel and the at least one imaging sensor.

7. The endoscopic device of claim 1, wherein the laser pathway shifter includes at least one prism.

8. The endoscopic device of claim 1, wherein the laser pathway shifter includes at least one mirror.

9. The endoscopic device of claim 1, wherein the imaging pathway shifter includes at least one prism.

10. The endoscopic device of claim 1, wherein the imaging pathway shifter includes at least one mirror.

11. The endoscopic device of claim 1, wherein the illumination channel and the imaging channel are contained in a rigid scope channel and the illumination channel and/or the imaging channel includes at least one relay lens.

12. An endoscopic device, comprising:
an illumination channel;
an imaging channel parallel to the illumination channel and having a different longitudinal axis from a longitudinal axis of the illumination channel;
a laser diode at a proximal end of the illumination channel and offset from the longitudinal axis of the illumination channel and configured to emit a laser;
at least one imaging sensor at a proximal end of the imaging channel and offset from the longitudinal axis of the imaging channel;
a first pair of prisms between the laser diode and the proximal end of the illumination channel and configured to change a propagation direction of the laser; and
a second pair of prisms between the proximal end of the imaging channel and the at least one imaging sensor and configured to change a propagation direction of light from the imaging channel,
wherein the illumination channel is configured to transmit the laser from the laser diode to a distal end of the illumination channel, and
wherein the imaging channel is configured to transmit light from a distal end of the imaging channel to the at least one imaging sensor.

13. The endoscopic device of claim 12, wherein the at least one imaging sensor includes a near-infrared (NIR) imaging sensor.

14. The endoscopic device of claim 12, wherein the at least one imaging sensor includes a red-green-blue (RGB) imaging sensor.

15. The endoscopic device of claim 12, wherein the at least one imaging sensor includes a near-infrared (NIR) imaging sensor and a red-green-blue (RGB) imaging sensor.

16. The endoscopic device of claim 13, further comprising a beam splitter between the second pair of prisms and the at least one imaging sensor.

17. An endoscopic device, comprising:
an illumination channel;
an imaging channel parallel to the illumination channel and having a different longitudinal axis from a longitudinal axis of the illumination channel;
a laser diode at a proximal end of the illumination channel and offset from the longitudinal axis of the illumination channel and configured to emit a laser;
at least one imaging sensor at a proximal end of the imaging channel and offset from the longitudinal axis of the imaging channel;
a first pair of prisms between the laser diode and the proximal end of the illumination channel and configured to change a propagation direction of the laser; and
a second pair of prisms between the proximal end of the imaging channel and the at least one imaging sensor and configured to change a propagation direction of light from the imaging channel,
wherein the illumination channel is configured to transmit the laser from the laser diode to a distal end of the illumination channel,
wherein the imaging channel is configured to transmit light from a distal end of the imaging channel to the at least one imaging sensor, and
wherein the at least one imaging sensor includes a near infrared (NIR) sensor.

18. The endoscopic device of claim 17, wherein the at least one imaging sensor further includes a red-green-blue (RGB) imaging sensor.

19. The endoscopic device of claim 18, further comprising a beam splitter between the second pair of prisms and the at least one imaging sensor.

20. The endoscopic device of claim 17, further comprising
processing circuitry configured to identify variations in speckle patterns in the light received by the at least one imaging sensor, or
transmission circuitry configured to transmit data corresponding to the light received by the at least one imaging sensor to external processing circuitry configured to identify the variations in the speckle patterns in the light received by the at least one imaging sensor.
